(19) United States
(12) Patent Application Publication
(10) Pub. No.: US 2004/0025193 A1
(43) Pub. Date: Feb. 5, 2004

(54) Method for the rapid selection of homozygous primary cell lines for the production of transgenic animals by somatic cell nuclear transfer

(76) Inventors: Yann Echelard, Jamaica Plain, MA (US); Li-How Chen, Acton, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/619,055

(22) Filed: Jul. 14, 2003

Related U.S. Application Data
(60) Provisional application No. 60/400,344, filed on Aug. 1, 2002.

(51) Int. Cl. A01K 67/027

(52) U.S. Cl. 800/6; 800/18; 800/21

(57) ABSTRACT

The present invention provides for the production of homozygous primary cells that carry a specific transgenic integration of interest on both chromosomes by bypassing breeding. These cell lines can they be used for the accelerated production of homozygous transgenic animals by somatic cell nuclear transfer. The invention is thus useful in the production of transgenic ungulate animals capable of producing desired biopharmaceuticals in their milk at higher yield than a comparable heterzygote. By combining the selection techniques of the current invention with somatic cell nuclear transfer it can be applied to large animals, where there is a strong need to shorten the time to homozygosity.

### FIELD OF THE INVENTION

The present invention relates to improved methods for the development of primary cell lines homozygous for a desired transgene(s) useful in the production of transgenic animals through somatic cell nuclear transfer. In particular the current invention provides a method for the accelerated production of transgenic animals homozygous for a selected trait

### BACKGROUND OF THE INVENTION

The present invention relates generally to the field of somatic cell nuclear transfer (SCNT) and to the creation of desirable transgenic animals. More particularly, it concerns improved methods for selecting, generating, and propagating superior somatic cell-derived cell lines, homozygous for one or more desired transgenes, and using these transfected cells and cell lines to generate transgenic non-human mammalian animal species, especially for the production of ungulates. Typically these transgenic animals will be used for the production of molecules of interest, including biopharmaceuticals, antibodies and recombinant proteins that are the subject of the transgene(s) of interest.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, and are often too time-consuming, costly and unreliable to develop. Moreover, these processes are completely incapable of allowing a specific animal line from producing gene products, such as desirable protein therapeutics that are otherwise entirely absent from the genetic complement of the species in question (i.e., human or humanized plasma protein or other molecules in bovine milk).

The development of technology capable of generating transgenic animals provides a means for exceptional precision in the production of animals that are engineered to carry specific traits or are designed to express certain proteins or other molecular compounds of therapeutic, scientific or commercial value. That is, transgenic animals are animals that carry the gene(s) of interest that has been deliberately introduced into existing somatic cells and/or germline cells at an early stage of development. As the animals develop and grow the protein product or specific developmental change engineered into the animal becomes apparent, and is present in their genetic complement and that of their offspring.

At present the techniques available for the generation of transgenic domestic animals are inefficient and time-consuming typically producing a very low percentage of viable embryos, often due to poor cell line selection techniques or poor viability of the cells that are selected. Moreover, once transgenic animals are developed they typically take a significant amount of time to optimize expression levels of desirable biopharmaceuticals and/or develop a commercially viable herd.

According to the prior art, the generation of an animal homozygous for the transgenic integration would require that the first transgenic offspring be bred to generate a heterozygous offspring of the opposite sex (or several heterozygous offspring of both sexes could be generated simultaneously if the first animal is male). This would be followed by the mating of a heterozygous male with a heterozygous female wherein the chances of developing a desirable homozygous animal for one gene would be one in four. Other techniques such as superovulation, flushing, and embryo transfer could also be applied to increase the chances of generating homozygous offspring. However these approaches do not diminish the need for 2 successive breeding cycles, with the associated increased time-lines. For example, in bovines, if the first heterozygous transgenic animal is a female calf, following birth that animal will need 14-15 months to reach maturity, and additional 9 months gestation to generate a heterozygous offspring (male). This offspring will then need an additional year to be able generate semen, and then an additional 9 months before the birth of the homozygous offspring could be contemplated. A total of 3-4 years is then necessary for the birth of the homozygous animals. Similar timelines are present for other ungulates including goats or sheep.

During the development of a transgenic cells, DNA sequences are typically inserted at random into the genetic complement of the target cell nuclei, which can cause a variety of problems. The first of these problems is insertional inactivation, which is inactivation of an essential gene due to disruption of the coding or regulatory sequences by the incoming DNA potentially made lethal through homozygousity. Another problem is that the transgene may either be not incorporated at all, or incorporated but not expressed. A further problem is the possibility of inaccurate regulation or expression due to positional effects in the genetic material. That is, the integration of exogenous DNA can effect the overall level of transgene expression and/or the accuracy of gene regulation between different founder animals produced with the same transgenic constructs. Thus, it is not uncommon to generate a large number of founder animals and often confirm that less than 5% express the transgene in a manner that warrants the development and commercialization of that transgenic line.

Additionally, the efficiency of generating transgenic domestic animals is generally low, with efficiencies of 1 in 100 offspring generated being transgenic not uncommon (Wall, 1997). As a result the cost associated with generation of transgenic animals can be as much as ($500,000) five hundred thousand dollars per expressing animal (Wall, 1997).

Prior art methods of nuclear transfer and microinjection have typically used embryonic and somatic cells and cell lines selected without regard to any objective factors tying cell quality relative to the procedures necessary for transgenic animal production.

Thus although transgenic animals have been produced by various methods in several different species, methods to readily and reproducibly produce transgenic animals capable of expressing a desired protein or biopharmaceutical in high quantity or demonstrating the genetic alteration or enhancement caused by the insertion of the transgene(s) at reasonable costs are still lacking.

Accordingly, a need exists for improved methods of transgenic animal generation, especially in the generation of homozygous animals for any desired transgene to enhance the commercial value of such animals. The methods of the invention are typically applied to primary somatic cells, in the context of nuclear transfer, for the accelerated generation of a herd of homozygous transgenic animals useful in the production of recombinant proteins in milk.

### SUMMARY OF THE INVENTION

Briefly stated, the current invention provides a method for the accelerated production of transgenic animals homozygous for a selected trait. The method involves transfecting a non-human mammalian cell-line with a given transgene construct containing at least one DNA encoding a desired gene; selecting a cell line(s) in which the desired gene has been inserted into the genome of that cell or cell-line; performing a nuclear transfer procedure to generate a transgenic animal heterzygous for the desired gene; characterizing the genetic composition of the heterzygous transgenic animal; selecting cells homozygous for the desired transgene through the use of selective agents; characterizing surviving cells using known molecular biology methods; picking surviving cells or cell colonies cells for use in a second round of nuclear transfer or embryo transfer; and producing a homozygous animal for a desired transgene.

An additional step that may performed according to the invention is to expand the biopsied cell-line obtained from the heterozygous animal in cell and/or cell-line in culture. An additional step that may performed according to the invention is to biopsy the heterozygous transgenic animal.

Alternatively a nuclear transfer procedure can be conducted to generate a mass of transgenic cells useful for research, serial cloning, or in vitro use. In a preferred embodiment of the current invention surviving cells are characterized by one of several known molecular biology methods including without limitation FISH, Southern Blot, PCR. The methods provided above will allow for the accelerated production of herd homozygous for desired transgene(s) and thereby the more efficient production of a desired biopharmaceutical.

Alternatively, the current invention allows for the production of genetically desirable livestock or non-human mammals.

In an alternate embodiment of the current invention multiple proteins can be integrated into the genome of a transgenic cell line. Successive rounds of transfection with another the DNA for an additional gene/molecule of interest (e.g., molecules that could be so produced, without limitation, include antibodies, biopharmaceuticals). Additionally these molecules could utilize different promoters that would be actuated under different physiological conditions or would lead to production in different cell types. The beta casein promoter is one such promoter turned on during lactation in mammary epithelial cells, while other promoters could be turned on under different conditions in other cellular tissues.

In addition, the methods of the current invention will allow the accelerated development of one or more homozygous animals that carry a particularly beneficial or valuable gene, enabling herd scale-up and potentially increasing herd yield of a desired protein much mote quickly than previous methods. Likewise the methods of the current invention will also provide for the replacement of specific transgenic animals lost through disease or their own mortality. It will also facilitate and accelerate the production of transgenic animals constructed with a variety of DNA constructs so as to optimize the production and lower the cost of a desirable biopharmaceutical. In another objective of the current invention homozygous transgenic animals are more quickly developed for xenotransplantation purposes or developed with humanized Ig loci.

### BRIEF DESCRIPTION OF THE DRAWINGS

FIG. 1 Shows a flowchart of the methods involved in practicing the invention.

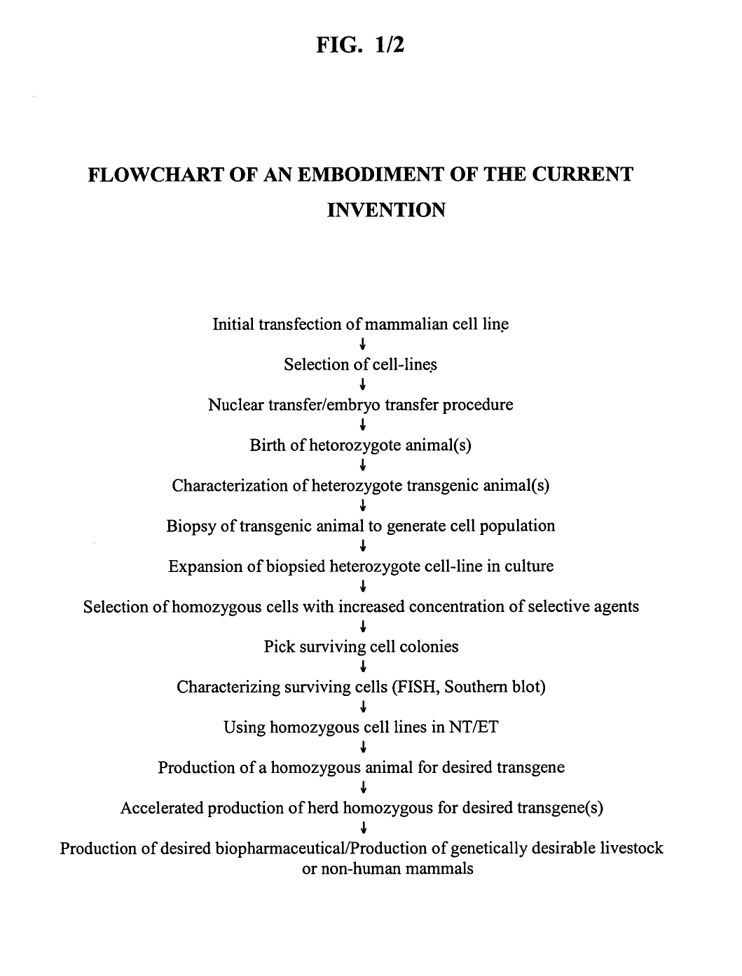

FIG. 2 Shows A Generalized Diagram of the Process of Creating Cloned Animals through Nuclear Transfer.

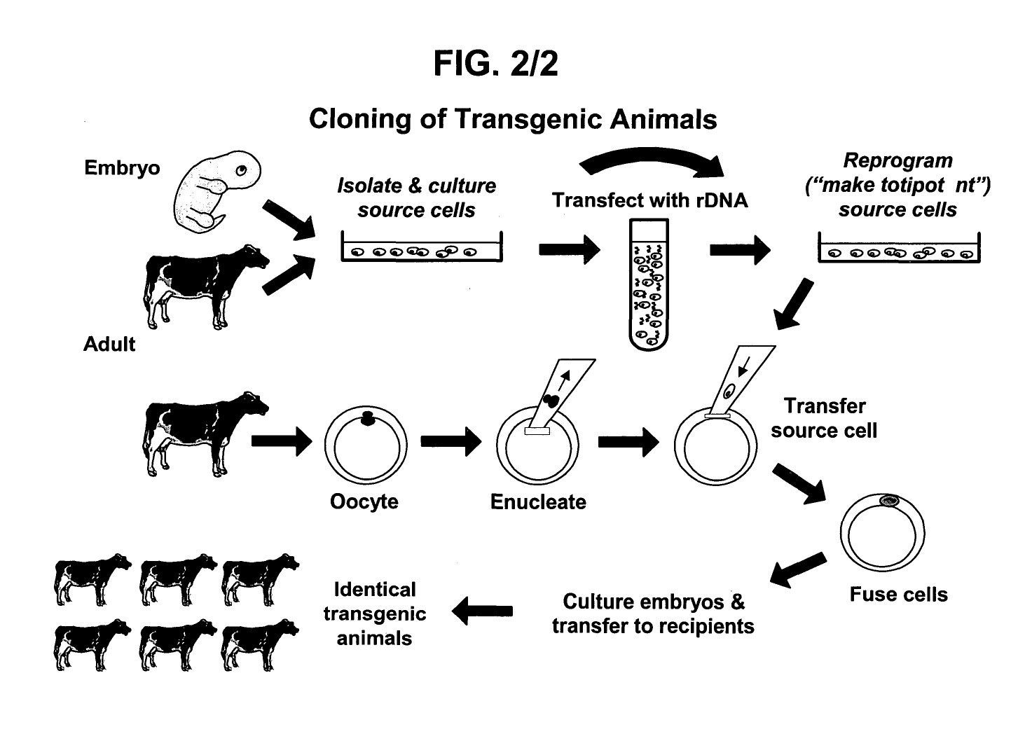

### DETAILED DESCRIPTION

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:

Bovine—Of or relating to various species of cows.

Caprine—Of or relating to various species of goats.

Cell Couplet—An enucleated oocyte and a somatic or fetal karyoplast prior to fusion and/or activation.

Cytocholasin-B—A metabolic product of certain fungi that selectively and reversibly blocks cytokinesis while not effecting karyokinesis.

Cytoplast—The cytoplasmic substance of eukaryotic cells.

Fusion Slide—A glass slide for parallel electrodes that are placed a fixed distance apart. Cell couplets are placed between the electrodes to receive an electrical current for fusion and activation.

Karyoplast—A cell nucleus, obtained from the cell by enucleation, surrounded by a narrow rim of cytoplasm and a plasma membrane.

Nuclear Transfer—or “nuclear transplantation” refers to a method of cloning wherein the nucleus from a donor cell is transplanted into an enucleated oocyte.

Ovine—of, relating to or resembling sheep.

Parthenogenic—The development of an embryo from an oocyte without the penetrance of sperm

Porcine—of, relating to or resembling swine or pigs

Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.

Selective Agent—Compounds, compositions, or molecules that can act as selection markers for cells in that they are capable of killing and/or preventing the growth of a living organism or cell not containing a suitable resistance gene. According to the current invention such agents include, without limitation, Neomycin, puromycin, zeocin, hygromycin, G418, gancyclovir and FIAU. Preferably, for the current invention increasing the dosage of the selective agent will kill all cell lines that only contain one integration site (e.g., heterozygous animals and/or cells).

Somatic Cell—Any cell of the body of an organism except the germ cells.

Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

Transgenic Organism—An organism into which genetic material from another organism has been experimentally transferred, so that the host acquires the genetic information of the transferred genes in its chromosomes in addition to that already in its genetic complement.

Ungulate—of or relating to a hoofed typically herbivorous quadraped mammal, including, without limitation, sheep, swine, goats, cattle and horses.

Xenotransplantation—any procedure that involves the use of live cells, tissues, and organs from one animal source, transplanted or implanted into another animal species (typically humans) or used for clinical ex-vivo perfusion

According to the present invention, the accelerated development of superior transgenic genotypes of mammals with improved efficiencies, characteristics, or enhanced biopharmaceutical production, including caprines and bovines, is provided. The current invention will allow the production and multiplication of adult animals with a known homozygous transgenic profile thereby enhancing the production and/or quality of biopharmaceuticals and accelerating the development of a herd of such animals. Progress will be enhanced, for example, in the success rates of generation of many important mammalian species including goats, rodents, cows and rabbits. That is, by natural breeding, in goats from the birth of an heterozygote, it will take a minimum of 2 years to obtain an homozygote; in cows, from the birth of an heterozygote it will take a minimum of 4 years to obtain an homozygote. With the preferred embodiment of the current invention, the production of homozygous transgenic goats can be limited to 7-8 months from the birth of a heterozygous animal; and 11-12 months in bovines. Likewise the development of other transgenic homozygous ungulates can also be similarly accelerated.

The methods of the current invention will potentially result in many identical offspring in a short period, decreasing overall costs involved and improving efficiencies.

In accordance with the methods of the current invention a transgenic primary cell line (from either caprine, bovine, ovine, porcine or any other non-human vertebrate origin) suitable for somatic cell nuclear transfer is created by transfection of the transgene(s) of interest (for example a mammary gland-specific transgene(s) targeting expression of a human therapeutic protein(s) to the mammary gland). The transgene(s) can either contain a selection marker (such as Neomycin, puromycin, zeocin, hygromycin or any other selectable marker) or be co-transfected with a cassette able to express the selection in marker in cell culture.

Following selection of recombinant colonies, cells are isolated and expanded, with aliquots frozen for long-term preservation according to procedures known in the field. The selected transgenic cell-lines can be characterized using standard molecular biology methods (PCR, Southern blotting, FISH). Cell lines carrying a transgene(s) of the appropriate copy number, generally with a single integration site (although the same technique could be used with multiple integration sites) can then be used as karyoplast donors in a somatic cell nuclear transfer protocol. Following nuclear transfer, and embryo transfer to a recipient animal, and gestation, live transgenic offspring are obtained. Typically this transgenic offspring carries only one transgene integration on a specific chromosome, the other homologous chromosome not carrying an integration in the same site. Hence the transgenic offspring is heterozygous for the transgene, maintaining the current need for at least two successive breeding cycles to generate a homozygous transgenic animal.

According to one embodiment of the current invention a technique is provided that allows an acceleration of the process involved in the production of homozygous transgenic animals. Following the birth of the first heterozygous offspring, a biopsy is performed and a primary cell line is derived from the first offspring. Aliquots of this cell line are then treated with increased doses of the selective agent that was used during the original transfection. Typically G418, but puromycin, hygromycin, zeocin, gancyclovir, FIAU, or any other agent able to kill cells in culture and for which a suitable resistance gene is available can be used. Increasing the dosage of the selective agent will kill all cell lines that only contain one integration sites (heterozygous) and permit to select cells that have 2 chromosomes with the integration (homozygous). Thereafter nuclear transfer techniques are utilized to generate additional animals that are homozygous for the desired trait with the animals developed for that gene being homozygous.

The mechanism for the transition from heterozygosity to homozygosity may be accomplished either by inter-chromosomal recombination or by deletion of the chromosome not carrying the integration, followed by the complete duplication of the integration-carrying chromosome. (Mortensen et al., 1993, Mol. Cell. Biol.). Following the increased selection, resistant colonies are genotyped (either by FISH or Southern blotting) to insure that the resulting cell line carries twice as many copies of the transgene and that both chromosome carry the integration. In addition karyotyping should be performed to insure that the cell line as the normal chromosomal complement.

### EXAMPLE 1

Protocol Using G418 selection:

I. Plate primary cells at 2×10⁵/10 cm petri dish.

II. Set up 2 petris for every concentration of G418. Optimum concentrations of G418 will vary from cell line to cell line, example:

1.2 “

1.5 “

2.0 “

2.5 “

3.0 ″

Add the drug at the same time you plate the cells. No need to let the cells settle down first.

III. Feed plates daily for the next five days with fresh medium+drug. After ˜5 days most of the cells will be dead, so feeding can be dropped back to every other day or so.

IV. Pick 6-24 of the best looking clones from the highest concentration of G418 onto 24-well wells.

V. Freeze and expand for DNA and karyotyping. Immobilize cells on filters for interphase FISH.

In another embodiment of the current invention, following the initial transfection, and isolation of the cell line, the cells be subjected immediately to increased selection to generate the homozygous cell line prior to generate an offspring.

Animals that are homozygous for the transgenic integration of a defined biopharmaceutical or stably carrying a desirable trait are beneficial for several reasons. First, this permits to potentially double the output of the transgenic animal. It also greatly simplifies and reduces the cost of expanding a herd of transgenic animals, since heterozygous animal will only transmit a specific transgenic integration site to only half of their offspring, whereas homozygous animals will transmit it to all their offspring. Other potential advantages are evident in cases where the transgene integration is targeted to a specific locus (for example an endogenous immunoglobulin locus) and that the ultimate objective is to inactivate both copy of that locus.

The advantage of this method is that it permits the generation of homozygous transgenic animals by bypassing 2 generations of breeding. Homozygous animal have the advantage of potentially doubling the production due to the transgene. For example, a heterozygous does belonging the “zygote” goat transgenic line and carrying only one chromosome with a the transgenic integration, were shown to produce a commercial antibody at the rate of 1 gram/per liter in their milk. Following breeding, homozygous females were obtained, carrying 2 transgenic chromosomes. For the homozygous does, the yield of the commercial antibody was 2 grams/liter of milk (double than the heterozygous does).

Experiments:

This general approach has been used with embryonic stem cells and primary fibroblasts in mice and rats, to speed up gene targeting. In this situation, blastocyst injection was used to generate animals from the selected embryonic stem cells. The originality of the invention is this general strategy of increasing the selective pressure to select homozygous cell line is now to be combined with somatic cell nuclear transfer in the generation of transgenic large animals. In this case, the aim is mostly to speed the generation of valuable large animal to be used in the production of therapeutic proteins.

In addition, the present invention relates to cloning procedures in which cell nuclei derived from somatic or differentiated fetal or adult mammalian cell lines are utilized. These cell lines include the use of serum starved differentiated fetal or adult caprine or bovine (as the case may be) cell populations and cell lines later reintroduced to serum as mentioned infra, these cells are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al, 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al., Biol. Reprod. 2001; Lai et al., 2001; Yong et al., 1998; and Kasinathan et al., Nature Biotech. 2001).

Materials And Methods

Estrus synchronization and superovulation of donor does used as oocyte donors, and micro-manipulation was performed as described in Gavin W. G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al, 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Enucleation and reconstruction was performed with, but may also be performed without, staining the oocytes with Hoechst 3342 at approximately 0.1-5.0 ug/ml and ultraviolet illumination of the genetic material/metaphase plate. Following enucleation and reconstruction, the karyoplast/cytoplast couplets were incubated in equilibrated Synthetic Oviductal Fluid medium supplemented with fetal bovine serum (1% to 15%) plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

Fusion was performed using a fusion slide constructed of two electrodes. The fusion slide was placed inside a fusion dish, and the dish was flooded with a sufficient amount of fusion buffer to cover the electrodes of the fusion slide. Cell couplets were removed from the culture incubator and washed through fusion buffer. Using a stereomicroscope, cell couplets were placed equidistant between the electrodes, with the karyoplast/cytoplast junction parallel to the electrodes. In these experiments an initial single simultaneous fusion and activation electrical pulse of approximately 2.0 to 3.0 kV/cm for 20 (can be 20-60) μsec was applied to the cell couplets using a BTX ECM 2001 Electrocell Manipulator. The fusion treated cell couplets were transferred to a drop of fresh fusion buffer. Fusion treated couplets were washed through equilibrated SOF/FBS, then transferred to equilibrated SOF/FBS with (1 to 10 μg/ml) or without cytochalasin-B. The cell couplets were incubated at 37-39° C. in a humidified gas chamber containing approximately 5% CO₂ in air.

Starting at approximately 30 minutes post-fusion, karyoplast/cytoplast fusion was determined. Fused couplets received an additional single electrical pulse (double pulse) of approximately 2.0 kV/cm for 20 (20-60) μsec starting at 1 hour (15 min-1 hour) following the initial fusion and activation treatment to facilitate additional activation. Alternatively, another group of fused cell couplets received three additional single electrical pulses (quad pulse) of approximately 2.0 kV/cm for 20 μsec, at fifteen-minute intervals, starting at 1 hour (15 min to 1 hour) following the initial fusion and activation treatment to facilitate additional activation. Non-fused cell couplets were refused with a single electrical pulse of approximately 2.6 to 3.2 kV/cm for 20 (20-60) μsec starting at 1 hours following the initial fusion and activation treatment to facilitate fusion. All fused and fusion treated cell couplets were returned to SOF/FBS with (1 to 10 μg/ml) or without cytochalasin-B. The cell couplets were incubated at least 30 minutes at 37-39° C. in a humidified gas chamber containing approximately 5% CO₂ in air.

Starting at 30 minutes following re-fusion, the success of karyoplast/cytoplast re-fusion was determined. Fusion treated cell couplets were washed with equilibrated SOF/FBS, then transferred to equilibrated SOF/FBS with (1 to 10 μg/ml) or without cycloheximide. The cell couplets were incubated at 37-39° C. in a humidified gas chamber containing approximately 5% CO₂ in air for up to 4 hours.

Following cycloheximide treatment, cell couplets were washed extensively with equilibrated SOF medium supplemented with bovine serum albumin (0.1% to 1.0%) plus 100 U/ml penicillin and 100 μg/ml streptomycin (SOF/BSA). Cell couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

The ability to pre-select a superior cell line to be used in a nuclear transfer program has remarkable implications. A significant amount of nuclear transfer work occurs with limited success as seen by the publications referenced in this document. In many of these publications a fair amount of work is done with very poor results or a complete lack of offspring born for individual cell (karyoplast) lines.

Paramount to the success of any nuclear transfer program is having adequate fusion of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both fusion and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

Goats.

The herds of pure- and mixed-breed scrapie-free Alpine, Saanen and Toggenburg dairy goats used as cell and cell line donors for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) for at least 6 hours prior to fusion to the enucleated oocytes.

Oocyte Collection.

Oocyte donor does were synchronized and superovulated as previously described (Gavin W. G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

All nuclear transfer embryos were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2μ (Lutalyse, Upjohn). Parturition occurred within 24 hours after treatment. Kids were removed from the dam immediately after birth, and received heat-treated colostrum within 1 hour after delivery.

Genotyping of Cloned Animals.

Shortly after birth, blood samples and ear skin biopsies were obtained from the cloned female animals (e.g., goats) and the surrogate dams for genomic DNA isolation. According to the current invention each sample may be first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKemg®, Me.) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with ³²P dCTP using the Prime-It® kit (Stratagene, La Jolla, Calif.). Hybridization was executed at 65° C. overnight. The blot was washed with 0.2 X SSC, 0.1% SDS and exposed to X-OMAT™ AR film for 48 hours.

The present invention allows for increased efficiency of transgenic procedures by increasing the number of potentially useful transgenic lines. Since it allows the rapid generation of transgenic animals with double the yield of recombinant protein production. Moreover, expansion of a transgenic herd from homozygote females will be more efficient since all the offspring will be transgenic.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, hormones, plasma proteins, and other molecules of interest in the milk or other bodily fluid (i.e., urine or blood) of transgenic animals homozygous for a desired gene. Proteins capable of being produced in through the method of the invention include: antithrombin III, lactoferrin, urokinase, PF4, alpha-fetoprotein, alpha-1-antitrypsin, C-1 esterase inhibitor, decorin, interferon, ferritin, prolactin, CFTR, blood Factor X, blood Factor VIII, as well as monoclonal antibodies.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds.

Accordingly, it is to be understood that the embodiments of the invention herein providing for an increased efficiency and speed in the production of transgenic animals are merely illustrative of the application of the principles of the invention. It will be evident from the foregoing description that changes in the form, methods of use, and applications of the elements of the disclosed method for the improved selection of cell or cell lines for use in nuclear transfer or micro-injection procedures to develop cell lines homozygous for a given gene(s) are novel and may be modified and/or resorted to without departing from the spirit of the invention, or the scope of the appended claims.

### LITERATURE CITED AND INCORPORATED BY REFERENCE

1. Alberio R, et al., (2001). Mammalian Oocyte Activation: Lessons from the Sperm and Implications for Nuclear Transfer, INT J DEV BIOL 2001; 45: 797-809.

2. Arroyo, A. et al., (2000). In Vivo Roles Of Integrins During Leukocyte Development And Traffic: Insights From The Analysis Of Mice Chimeric For α₅, α_{V}, and α₄ Integrins. THE JI 165: 4667-4675.

3. Baguisi A,(1999) et al., Production of Goats by Somatic Cell Nuclear Transfer, NAT BIOTECH; 17: 456-461.

4. Byrd, N, et al., (2002). Hedgehog Is Required For Murine Yolk Sac Angiogenesis. DEVELOPMENT 129: 361-372.

5. Cibelli JB, (1998) et al., Cloned Transgenic Calves Produced From Nonquiescent Fetal Fibroblasts. SCIENCE; 280: 1256-1258.

6. Helgason, C et al., (1998). Targeted Disruption Of SHIP Leads To Hemopoietic Perturbations, Lung Pathology, And A Shortened Life Span. GENES & DEV. 12: 1610-1620.

7. Hu, J.-F, et al., (1997). Genomic Deletion Of An Imprint Maintenance Element Abolishes Imprinting Of Both Insulin-Like Growth Factor II And H19. J. BIOL. CHEM. 272: 20715-20720.

8. Hwang, H.-Y, et al., (1996). Creation Of Homozygous Mutants Of Leishmania Donovani With Single Targeting Constructs. J. BIOL. CHEM. 271: 30840-30846.

9. Jain, M, et al., (2001). Targeted Inactivation Of GaI Does Not Alter Cardiac Function Or β-Adrenergic Sensitivity. AM. J. PHYSIOL. 280: 569H-575.

10. Kasinathan P, (2001) et al., Effect of Fibroblast Donor Cell Age and Cell Cycle on Development of Bovine Nuclear Transfer Embryos In Vitro, BIOL REPROD.; 64(5): 1487-1493.

11. Kasinathan P, (2001) et al., Production of Calves from G1 Fibroblasts, NATURE BIOTECH; 19: 1176-1178.

12. Klug, M, et al., (1996). Genetically Selected Cardiomyocytes From Differentiating Embryonic Stem Cells Form Stable Intracardiac Grafts. J. CLIN. INVEST. 98: 216-224.

13. Koike, H, et al., (2002). Efficient Biallelic Mutagenesis With Cre/Loxp-Mediated Inter-Chromosomal Recombination. EMBO REPORTS 3: 433-437.

14. Kundu, R et al., (1998). Targeted Inactivation Of The Coagulation Factor IX Gene Causes Hemophilia B In Mice. BLOOD 92: 168-174.

15. Lebel, M, et al., (1998). A Deletion Within The Murine Werner Syndrome Helicase Induces Sensitivity To Inhibitors Of Topoisomerase And Loss Of Cellular Proliferative Capacity. PROC. NATL. ACAD. SCI. U.S.A. 95: 13097-13102.

16. Lim, S, et al., (1997). A Shortened Life Span Of EKLF-/-Adult Erythrocytes, Due To A Deficiency Of Beta-Globin Chains, Is Ameliorated By Human Gamma-Globin Chains. BLOOD 90: 1291-1299.

17. Mortensen, R, et al., (1992) Production Of Homozygous Mutant ES Cells With A Single Targeting Construct. MOL. CELL. BIOL. 12, 2391-2395.

18. Nagy, A, et al., (1996). Targeted Mutagenesis: Analysis Of Phenotype Without Germ Line Transmission. J. CLIN. INVEST. 97: 1360-1365.

19. Qu, C.-K, et al., (1998). Biased Suppression Of Hematopoiesis And Multiple Developmental Defects In Chimeric Mice Containing Shp-2 Mutant Cells. MOL. CELL. BIOL. 18: 6075-6082.

20. Sakai, E, et al., (1999). Recombination And Transcription Of The Endogenous Ig Heavy Chain Locus Is Effected By The Ig Heavy Chain Intronic Enhancer Core Region In The Absence Of The Matrix Attachment Regions. PROC. NATL. ACAD. SCI. U.S.A. 96: 1526-1531.

21. Saleque, S, et al., (2002). The Zinc-Finger Proto-Oncogene Gfi-1b Is Essential For Development Of The Erythroid And Megakaryocytic Lineages. GENES & DEV. 16: 301-306.

22. Soudais, C, et al., (1995). Targeted Mutagenesis Of The Transcription Factor GATA-4 Gene In Mouse Embryonic Stem Cells Disrupts Visceral Endoderm Differentiation In Vitro. DEVELOPMENT 121: 3877-3888.

23. Sowell, M et al., (1997). Targeted Inactivation Of Alpha I2 Or Alpha I3 Disrupts Activation Of The Cardiac Muscarinic K+ Channel, IK+Ach, In Intact Cells. PROC. NATL. ACAD. SCI. U.S.A. 94: 7921-7926.

24. Stanford, W, et al., (1998). Expression Trapping: Identification Of Novel Genes Expressed In Hematopoietic And Endothelial Lineages By Gene Trapping In ES Cells. BLOOD 92: 4622-4631.

25. Weiss, M and Orkin, S (1996). In Vitro Differentiation Of Murine Embryonic Stem Cells. New Approaches To Old Problems. J. CLIN. INVEST. 97: 591-595.

26. Wu, Q, et al., (1998). Generation And Characterization Of Mice Deficient In Hepsin, A Hepatic Transmembrane Serine Protease. J. CLIN. INVEST. 101: 321-326.

27. Yong Z and L Yuqiang, (1998) Nuclear-Cytoplasmic Interaction and Development of Goat Embryos Reconstructed by Nuclear Transplantation: Production of Goats by Serially Cloning Embryos, BIOL REPROD.; 58: 266-269.

28. Zou X, et al.,(2002) Generation Of Cloned Goats (Capra Hircus) From Transfected Foetal Fibroblast Cells, The Effect Of Donor Cell Cycle, MOL REPROD DEV.; 61: 164-172.

What is claimed is:

1. A method for the accelerated production of transgenic animals homozygous for a selected trait comprising:
transfecting a non-human mammalian cell-line with a given transgene construct containing at least one DNA encoding a desired gene;
selecting a cell line(s) in which the desired gene has been inserted into the genome of that cell or cell-line;
performing a first nuclear transfer procedure to generate a first transgenic animal heterzygous for the desired gene;
characterizing the genetic composition of said first heterzygous transgenic animal;
selecting cells homozygous for the desired transgene through the use of a selective agent;
characterizing surviving cells using known molecular biology methods; and
picking surviving cells or cell colonies cells for use in a second round of nuclear transfer or embryo transfer; and producing a second transgenic animal homozygous for a desired transgene.

2. The method of claim 1, wherein said first transgenic animal is biopsied so as to characterize the genome of said first transgenic animal.

3. The method of claim 2, wherein the cells or cell line biopsied from said first transgenic animal is expanded through cell culture techniques.

4. The method of claim 1, wherein said surviving cell are characterized by one of several known molecular biology methods including without limitation FISH, Southern Blot, PCR.

5. The method of claim 1, wherein homozygous transgenic animals are more quickly developed for xenotransplantation purposes or developed with humanized Ig loci.

6. The method of claim 1, wherein said donor differentiated mammalian cell to be used as a source of donor nuclei or donor cell nucleus is from an ungulate.

7. The method of either claims 1 or 6, wherein said donor cell or donor cell nucleus is from an ungulate selected from the group consisting of bovine, ovine, porcine, equine, caprine and buffalo.

8. The method of claim 1, wherein said donor differentiated mammalian cell to be used as a source of donor nuclei or donor cell nucleus is from an adult non-human mammalian somatic cell.

9. The method of claim 1, wherein said non-human mammal is a rodent.

10. The method of claim 1, wherein said donor differentiated mammalian cell to be used as a source of donor nuclei or donor cell nucleus is a non-quiescent somatic cell or a nucleus isolated from said non-quiescent somatic cell.

11. The method of either claims 1 or 6, wherein the fetus develops into an offspring.

12. The resultant offspring of the methods of claim 1.

13. The resultant offspring of claim 1 further comprising wherein the offspring created as a result of said nuclear transfer procedure is homozygous for more than one desired gene.

14. The method of claim 1 further comprising using a second selective agent.

15. The method of claim 14 such that the transgenic homozygous cell lines selected can proceed through a second or more multiple rounds selection to generate a cell line homozygous for more than one desired gene.

16. The method of claim 1, wherein cytocholasin-B is used in the cloning protocol.

17. The method of claim 1, wherein cytocholasin-B is not used in the cloning protocol.

18. The method of claim 1, wherein said donor differentiated mammalian cell to be used as a source of donor nuclei or donor cell nucleus is a non-quiescent somatic cell or a nucleus isolated from said non-quiescent somatic cell.

19. The resultant offspring of the methods of claims 1 or 18.

20. The method of claim 1, wherein the techniques used to generate a homozygous cell line are used to develop a functional organ for transplantation.

21. The method of claim 20, wherein said cultured inner cell mass cells are used in organogenesis.

22. The method of claim 1 wherein the desired gene codes for a biopharmaceutical protein product.

23. The method of claim 22 wherein said biopharmaceutical protein product is a compound selected from the group consisting of: antithrombin III, lactoferrin, urokinase, PF4, alpha-fetoprotein, alpha-1-antitrypsin, C-1 esterase inhibitor, decorin, interferon, ferritin, transferrin conjugates with biologically active peptides or fragments thereof, human serum albumin, prolactin, CFTR, blood Factor X, blood Factor VIII, as well as monoclonal antibodies.

24. The method of claim 1 wherein the DNA construct containing the desired gene is actuated by at least one beta casein promoter.

25. The resultant milk derived from the offspring of the methods of claim 1 or 24.
